(19) United States
(12) Patent Application Publication
(10) Pub. No.: US 2025/0387671 A1
(43) Pub. Date: Dec. 25, 2025

(54) ELECTRONIC DEVICE, INFORMATION DISPLAY METHOD, AND RECORDING MEDIUM

(71) Applicant: CASIO COMPUTER CO., LTD., Shibuya-ku, Tokyo (JP)

(72) Inventor: Nobuteru TAKAHASHI, Tokyo (JP)

(73) Assignee: CASIO COMPUTER CO., LTD., Shibuya-ku, Tokyo (JP)

(21) Appl. No.: 19/226,472

(22) Filed: Jun. 3, 2025

(30) Foreign Application Priority Data
Jun. 21, 2024 (JP) 2024-100162

(51) Int. Cl. A63B 24/00; G06F 3/01; G16H 20/30; G16H 40/67

(52) U.S. Cl. CPC A63B 24/0075; G06F 3/011; G16H 20/30; G16H 40/67; A63B 2024/0081

(57) ABSTRACT

An electronic device to be equipped by a user for acquiring data related to exercise of the user includes an acquirer to acquire a measurement result that is a result of measurement of each of indicator values of a plurality of indicators related to the exercise, a storage to store the measurement result acquired by the acquirer, a display to display information based on the measurement, and a controller to, in response to start of new measurement, based on one or more indicator values that are included in the indicator values of the plurality of indicators included in the measurement result of most recent measurement stored in the storage and that satisfy a predetermined condition, determine a combination of two or more indicators of the plurality of indicators that are to be displayed on the display.

### CROSS-REFERENCE TO RELATED APPLICATIONS

This application claims the benefit of Japanese Patent Application No. 2024-100162, filed on Jun. 21, 2024, the entire disclosure of which is incorporated by reference herein.

### FIELD OF THE INVENTION

The present disclosure relates to an electronic device, an information display method, and a recording medium.

### BACKGROUND OF THE INVENTION

An exercise assistance device is under development that analyzes exercise data obtained when a user takes exercise such as running or walking and that outputs advice to the user. For example, Unexamined Japanese Patent Application Publication No. 2020-5766 discloses a running support system that displays a viewpoint for improvement in the level of running skills through a comparison with exercise data of past running with respect to the pros and cons of the running skills of a user.

### SUMMARY OF THE INVENTION

An electronic device according to an aspect of the present disclosure is an electronic device to be equipped by a user for acquiring data related to exercise of the user includes an acquirer to acquire a measurement result that is a result of measurement of each of indicator values of a plurality of indicators related to the exercise, a storage to store the measurement result acquired by the acquirer, a display to display information based on the measurement, and a controller to, in response to start of new measurement, based on one or more indicator values that are included in the indicator values of the plurality of indicators included in the measurement result of most recent measurement stored in the storage and that satisfy a predetermined condition, determine a combination of two or more indicators of the plurality of indicators that are to be displayed on the display.

### BRIEF DESCRIPTION OF DRAWINGS

A more complete understanding of this application can be obtained when the following detailed description is considered in conjunction with the following drawings, in which:

FIG. 1 is a block diagram illustrating functional configuration of an electronic device according to an embodiment;

FIG. 2 is a diagram showing example indicators;

FIG. 3 illustrates an example of configuration of a form analysis result screen;

FIG. 4 is a flowchart of indicator extraction processing according to the embodiment;

FIG. 5 is a flowchart of indicator display processing according to the embodiment;

FIG. 6 illustrates an example of a screen displaying improvement-recommended indicators; and

FIG. 7 illustrates an example of a normal screen with no improvement-recommended indicator.

### DETAILED DESCRIPTION OF THE INVENTION

Hereinafter, an electronic device and the like according to an embodiment are described while referencing the drawings. Note that, in the drawings, identical or corresponding components are denoted with the same reference numerals.

An electronic device 100 according to the embodiment is a wearable device that is worn by a user when exercising. An objective of the present embodiment is to extract and display, while the user is exercising, a high-priority indicator among indicators that need improvement. Although where on a body to wear the electronic device 100 depends on a type of the electronic device 100, in general, a waist-mounted type is desirable in the case where the measurement accuracy of exercise data is important, and an arm-mounted type is desirable in the case where the ease of viewing displayed information is important. The electronic device 100 analyzes exercise data of the user to obtain values of various indicators, calculates score reflecting an evaluation of the exercise of the user based on these values, and notifies the user an indicator with a value to be improved to achieve effective improvement in the score. Details of such operations of the electronic device 100 is described later.

The electronic device according to the present embodiment enables extracting and displaying, while the user is exercising, a high-priority indicator among indicators that need improvement.

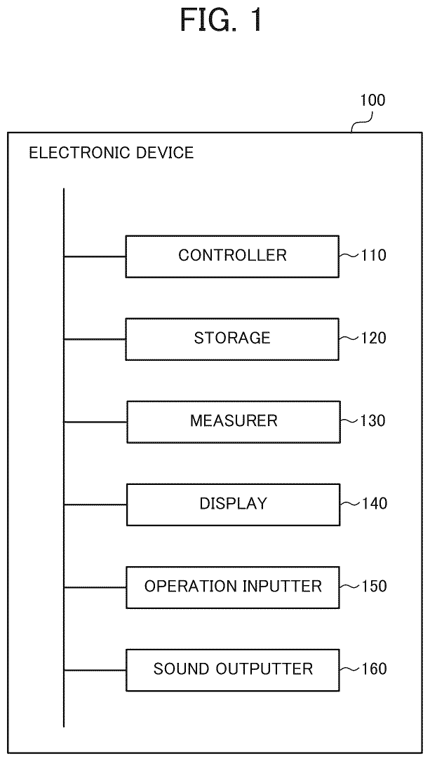

As illustrated in FIG. 1, the electronic device 100 functionally includes a controller 110, a storage 120, a measurer 130, a display 140, an operation inputter 150, and a sound outputter 160.

The controller 110 includes, for example, a processor, such as a central processing unit (CPU). The controller 110 executes, with a program stored in the storage 120, processing for achieving various functions of the electronic device 100, indicator extraction processing described later, indicator display processing described later, and other related processing.

The storage 120 stores the program to be executed by the controller 110 and necessary data such as a measurement result that is a result of the measurement by the measurer 130. The storage 120 may include random access memory (RAM), read-only memory (ROM), flash memory, or the like, but is not limited thereto. The storage 120 may be provided inside the controller 110.

The measurer 130 includes devices that detect various values related to exercise (running, walking, and the like) of the user. Examples of the devices include an acceleration sensor, a gyrosensor, a global positioning system (GPS) device, a heart rate sensor, a temperature sensor, a barometric sensor, and the like. The controller 110 can obtain the values measured by the respective devices included in the measurer 130 (measurement values) and derive an indicator value of each of the various indicators related to the exercise of the user. The controller 110 functions as an acquirer when acquiring the measurement result that is the result of the measurement by the measurer 130. The measurer 130 does not necessarily include all of the aforementioned sensors and thus does not need to include some of the sensors (such as the heart rate sensor, the temperature sensor, and the barometric sensor) if non-inclusion of such sensors does not hinder the derivation of the indicator values of the indicators related to the exercise of the user.

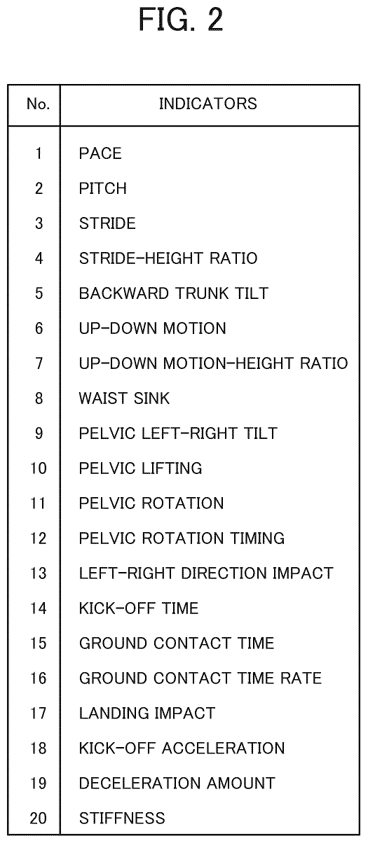

The controller 110 stores, in the storage 120, as exercise log data, the various measurement values as the values measured by the measurer 130, the various indicator values that are derived based on these various measurement values and that are values of the various indicators, and the various scores that are derived based on the various indicator values. The indicators are various types of measures for evaluation of exercise (for example, running) of the user, such as a pace (time required for one-kilometer running), pitch (number of steps per one minute), stride (distance traveled in one step from one ground contact to the next ground contact), and landing impact (amount of impact on a body immediately after landing), as in the examples of FIG. 2. The indicators include both an indicator for which the measurement value measured by the measurer 130 (the measurement result), as is, is simply taken to be the indicator value and an indicator for which the indicator value is derived based on multiple measurement values. Some of these indicators (such as the “pace”) may be used for determination of a target value (a value to aim for) of other indicator.

The display 140 includes a display such as a liquid crystal display or an organic electro-luminescence (EL) display. The display 140 displays information (various indicator values, form analysis results, and the like) based on the measurement by the measurer 130.

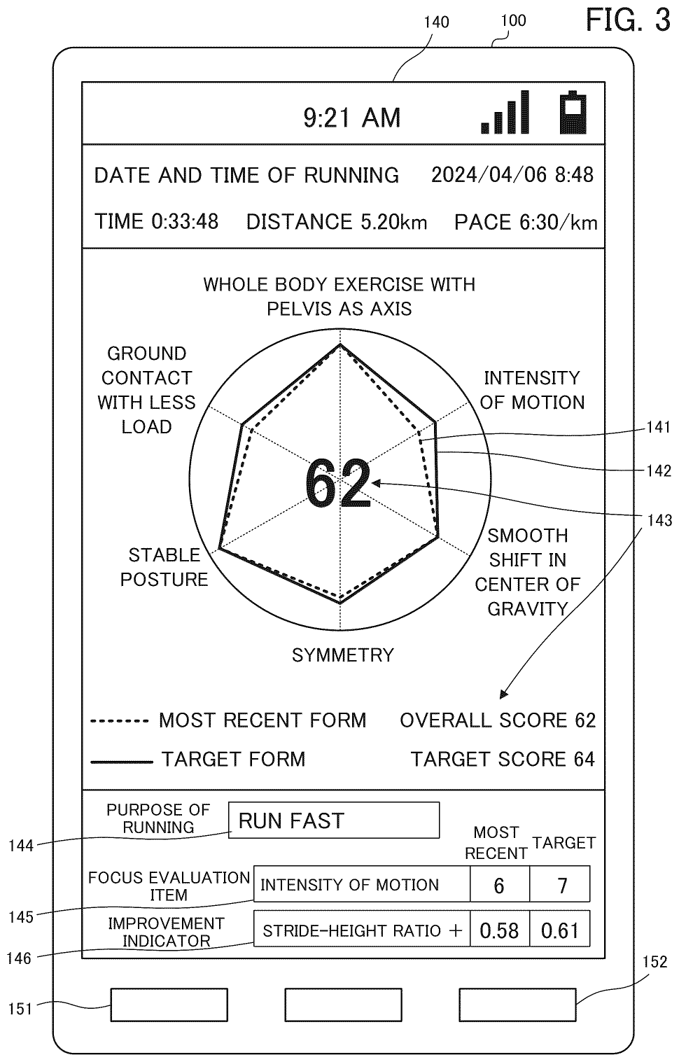

The operation inputter 150 is a user interface such as a push button switch, a touch panel, or the like, and receives operation inputs from the user. When the operation inputter 150 includes a touch panel, the touch panel may be implemented as a touch panel that is integrated with the display of the display 140. In the present embodiment, the operation inputter 150 includes push button switches such as an exercise start button 151 for notification to the electronic device 100 of the start of exercise of the user and an exercise end button 152 for notification to the electronic device 100 of the end of the exercise of the user, as illustrated in FIG. 3.

The sound outputter 160 includes sound output means such as a speaker or earphones and outputs voice announcements, notification sounds, sound effects, and the like. The exercise start button 151, the exercise end button 152, and the like, may arranged as the operation inputter 150 around the earphones. In the case where output of voice announcements, notification sounds, and the like, is not required, the electronic device 100 need not include the sound outputter 160.

The functional configuration of the electronic device 100 is described above. The controller 110 performs form analysis based on the measurement values (the exercise data of the user) measured by the measurer 130 and, as illustrated in FIG. 3, for example, displays radar charts 141 and 142, an overall score 143, and the like, based on various evaluation items (FIG. 3 illustrates an example in which the evaluation items are the following six evaluation items, “whole body exercise with a pelvis as an axis,” “ground contact with less load”, “intensity of motion,” “stable posture”, “smooth shift in the center of gravity”, and “symmetry”). Each of the evaluation items constituting the radar charts 141 and 142 is assigned one or more indicators related to the evaluation item (related indicators). A score of each of the evaluation items (evaluation item score) is calculated by obtaining a weighted sum of scores of the corresponding related indicators derived from the indicator values of these indicators (indictor scores of the related indicators), and the radar charts 141 and 142 are drawn based on the evaluation item scores. Additionally, the overall score 143 is calculated by obtaining a weighted sum of the evaluation item scores depending on a purpose of exercise (purpose of running). However, the aforementioned methods for calculating each of the evaluation item scores and the overall score 143 are merely examples, and these scores may be calculated using other methods.

Although the method for deriving each of the indictor scores can also be selected freely, some methods are described below.

For example, a first method may be used that includes referencing a predefined table. This is a method of deriving, for each of the indicators, the indictor score of the indicator based on the indicator value by referencing a table (indictor score definition table) in which information such as “if the indicator value is between x and y, the score is z” is specified for each user profile information (especially the value of the indicator “pace”).

Furthermore, a second method may be used that includes referencing a more simplified table. This is a method of deriving, for each of the indicators, the indictor score of the indicator by obtaining a reference value of the indicator with reference to a table (indicator reference value table) in which ideal reference values (indicating a range of the indicator value with which a perfect score, such as 100 points, is achievable) is set for each of values corresponding to various paces of the user, and deducting points from the full score based on an extent of deviation of the indicator value from the reference values.

Furthermore, a third method may be used that is a method of deriving, for each of the indicators, the indictor score of the indicator by calculating the ideal reference values of the indicator (indicating a range of the indicator value with which a perfect score is achievable) for each of values corresponding to various paces of the user by a math formula without using a table, and deducting points from the full score based on an extent of deviation of the indicator value from the reference values.

The aforementioned three methods for deriving the indictor score are mere examples, and the method for deriving the indictor score is not limited to those methods.

FIG. 3 illustrates not only the radar chart 141 that is based on the form of the most recent exercise, but also the radar chart 142 that is based on a target form (the form in the case of running in a target running style). This radar chart 142 is obtained with the evaluation item scores that are each calculated from the indictor score obtained as a result of improvement in a value of the corresponding indicator in accordance with the purpose of exercise (purpose of running).

As described above, the overall score 143 is calculated by obtaining a weighted sum of the evaluation item scores depending on the purpose of exercise (purpose of running). The purpose of exercise (purpose of running) is set by the user when using the electronic device 100, and is a setting of what the user wishes to achieve by exercising (running), such as “run fast”, “run long”, “run for health”, “run with ease”, and the like. Once the user has set the purpose of exercise, presenting information to the user is desirable that indicates a type of the indicator to be improved for achievement of the purpose. Thus, the storage 120 pre-stores an improvement-recommended indicator candidate table including information that associates each of various purposes with candidates (improvement-recommended indicator candidates) of an indicator to be improved for achievement of the purpose (improvement-recommended indicator). Additionally, information of the evaluation item score to be focused on for achievement of a set purpose is useful for the user. Thus, the storage 120 may pre-store a focus-recommended evaluation item table including information that associates each of various purposes with an evaluation item to be focused on for achievement of the purpose (focus-recommended evaluation item). In the present embodiment, the storage 120 is taken to store the improvement-recommended indicator candidate table and the focus-recommended evaluation item table.

For example, in the case where the purpose of running is “run for health”, since preventing injury is important, the indicator values of “landing impact”, “kick-off acceleration”, “up-down motion-height ratio” are to be smaller for a reduction in knee load. Accordingly, the indicators “landing impact”, “kick-off acceleration”, “up-down motion-height ratio”, and the like, are associated as the improvement-recommended indicator candidates with the purpose “run for health” and are stored in the improvement-recommended indicator candidate table. In the indicator extraction processing described below, among these improvement-recommended indicator candidates, an indicator that is considered to have the largest contribution to score improvement is extracted. Similarly, in this case, since “ground contact with less load” is the most important among the evaluation items constituting the radar chart 141, the focus-recommended evaluation item table stores the purpose “run for health” in association with the evaluation item “ground contact with less load”.

As understood from the above explanation, the improvement-recommended indicator candidates and the focus-recommended evaluation item differ depending on the purpose of running. Other examples of the improvement-recommended indicator candidates are described below. For example, in the case where the purpose is “run fast”, since the “stride-height ratio” is to be increased, the improvement-recommended indicator candidates are the “stride-height ratio” and the like. Other examples of the improvement-recommended indicator candidates are described below. For example, in the case where the purpose is “run long”, since the “kick-off acceleration” is to be decreased, the improvement-recommended indicator candidates are the “kick-off acceleration” and the like. Furthermore, as described above, the indicator to be improved may be an indicator to be increased for improvement or an indicator to be decreased for improvement (or may be an indicator “to be kept within a predetermined range”), and thus the improvement-recommended indicator candidate table may store such types of the indicators (“to be increased”, “to be decreased”, “to be kept within a predetermined range”, and the like) so as to enable displaying a mark or the like indicating a type of the types. One example according to this configuration is to display “+” for “an indicator for which larger is better”, “−” for “an indicator for which smaller is better”, and “=” for “an indicator to be kept within a predetermined range”. The “+” of the “stride-height ratio+” in a display area 146 of FIG. 3 indicates that the stride-height ratio is “an indicator for which larger is better”. The examples of the improvement-recommended indicator candidate described here are mere examples, and two or more improvement-recommended indicator candidates may be associated with each of various purposes.

As a result of performing the form analysis after exercise of the user, the controller 110 sets a target value of each indicator based on the user profile information (height, weight, gender, pace, and the like) and the exercise log data. For example, the target value may be set by referencing a table (target value setting table) created in advance by collecting information such as “at this pace, this is the level of value you should aim for,” or may be set by calculating a value that is likely to be achievable by the user without difficulty based on previous exercise log data.

FIG. 3 illustrates a display example in the case where “run fast” is set as the purpose of running as indicated in a display area 144. The “stride-height ratio” displayed in the display area 146 as the improvement-recommended indicator (“improvement indicator” in FIG. 3) is extracted in the indicator extraction processing that is described later. The “intensity of motion” displayed in a display area 145 as the focus-recommended evaluation item (“focus evaluation item” in FIG. 3) is acquired with reference to the focus-recommended evaluation item table. FIG. 3 further illustrates displaying “0.58” as the most recent indicator value and “0.61” as the target value in the display area 146 and displaying “6” as the most recent score and “7” as a target score in the display area 145. This means that the indicator value of the “stride-height ratio” of the most recent exercise is “0.58” and the evaluation item score of the “intensity of motion” derived based on the related indicators including this indicator value is “6”, and that the target value of the “stride-height ratio” based on the most recent pace of the user is “0.61” and an increase in the indicator value of the “stride-height ratio” from “0.58” in the most recent exercise to “0.61” in the next exercise increases the evaluation item score of the “intensity of motion” to “7”. FIG. 3 further illustrates that the overall score 143 is “62” and the target score is “64”. This indicates that the increase in the indicator value of the “stride-height ratio” to “0.61” increases the evaluation item score of the “intensity of motion” to “7” and leads to improvement in the overall score 143 from the most recent score “62” to the target score “64”.

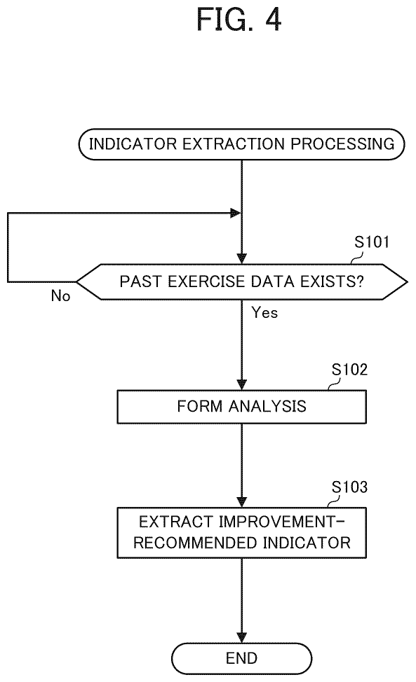

The indicator extraction processing that is processing for extracting the improvement-recommended indicator based on the exercise data in the past is described with reference to FIG. 4. This processing is executable at any timing but is normally executed after the end of exercise of the user wearing the electronic device 100.

Firstly, the controller 110 determines whether the storage 120 stores the exercise data in the past (step S101). When the exercise data in the past is not stored (step S101; No), the processing returns to step S101. When the exercise data in the past is stored (step S101; Yes), the controller 110 performs the form analysis based on the most recent exercise data (step S102). In this step, the controller 110 derives the indictor score of each of the indicators based on the corresponding indicator value of the most recent exercise stored in the storage 120 as the exercise log data, calculates the evaluation item score of each of the multiple evaluation items for creation of the radar chart 141 by obtaining a weighted sum of the indictor scores of the related indicators of the evaluation item, and calculates the overall score 143 by obtaining a weighted sum of the evaluation item scores of the evaluation items depending on the purpose of running of the user. Additionally, for each of the indicators, the controller 110 calculates the target value of the indicator based on the exercise data in the past, and stores each of the indicator value, the target value, and the score that are obtained or calculated in this step through addition to the exercise log data in the storage 120.

Then the controller 110 extracts the improvement-recommended indicator (step S103) and ends the indicator extraction processing. In step S103, the controller 110 firstly acquires the improvement-recommended indicator candidates based on the improvement-recommended indicator candidate table stored in the storage 120 and the purpose of running of the user, and extracts, from these improvement-recommended indicator candidates, as the improvement-recommended indicator, a predetermined number of the indicators that are considered to have a larger contribution to the improvement of the overall score 143. The improvement-recommended indicator extracted in this step is not limited to only one indicator that is considered to have the largest contribution, and a predetermined number of the indicators may be selected in order from those with the highest contribution and extracted as the improvement-recommended indicators. Any number may be used as the number of the improvement-recommended indicators to be extracted, and a configuration may be used in which the number is freely changeable to a number desired by the user. Although the method for deriving from the improvement-recommended indicator candidates the improvement-recommended indicator, which is an indicator that is considered to have a larger contribution to score improvement, can also be selected freely, some methods are described below.

For example, a first method may be used in which the controller 110 references the exercise log data stored in the storage 120 for each of the indicators included in the improvement-recommended indicator candidates, determines for each of the indicators the differential coefficient of the indictor score corresponding to the value of the indicator of the most recent exercise, and extracts one or more improvement-recommended indicators in descending order of the differential. In the case where the indictor score is a non-differentiable function, such as the case of using the indictor score definition table for deriving the indictor score, extraction as the one or more improvement-recommended indicators may be performed in the order in which an increase in the indicator value for an increase (for example, an increase by 1) in the indictor score is smaller.

Furthermore, a second method, which can also be deemed as a modification of the first method, may be used in which the controller 110 determines a standard deviation for each of the indicators included in the improvement-recommended indicator candidates by referencing the exercise log data stored in the storage 120, calculates for each of the indicators an increase in the indictor score in response to addition or subtraction of a constant (for example, 0.1) multiple of the standard deviation to or from the indicator value of the most recent exercise, and extracts one or more improvement-recommended indicators in descending order of the calculated increase in the indictor score.

Furthermore, a third method may be used in which the controller 110 sets a target value of each of the indicators included in the improvement-recommended indicator candidates by performing the form analysis based on the exercise log data stored in the storage 120, and extracts one or more improvement-recommended indicators in descending order of a difference between the set target value and the indicator value of the most recent exercise.

Since the range of a change in the indicator value varies greatly depending on the type of indicator, to prevent the effect of this variation, the indicator values may be normalized using the exercise log data and the like, instead of using the indicator values directly. One example method for normalization of the indicator values is a method of subtracting from each of the indicator values a mean value of the corresponding indicator and divining the resultant value by the standard deviation of this indicator.

The controller 110 executes the indicator extraction processing described above to extract a predetermined number (for example, the number set by the user) of the improvement-recommended indicators. Furthermore, since the indicator value, the target value, and the indictor score of each of the indicators that are obtained as a result of the form analysis, and the evaluation item score and the target score of each of the evaluation items, are stored in the exercise log data, the controller 110 can cause the extracted improvement-recommended indicator and the most recent indicator value and the target value that correspond to this improvement-recommended indicator to be displayed in the display 140, as in the display area 146 of FIG. 3. Furthermore, the controller 110 can cause, based on the focus-recommended evaluation item table stored in the storage 120 and the exercise log data, the type of the focus-recommended evaluation item and the most recent score and the target score that correspond to this focus-recommended evaluation item to be displayed in the display 140, as in the display area 145 of FIG. 3. The target score is a score corresponding to the case where a target value of each value of the related indicators assigned to the corresponding evaluation item is achieved, and is calculated based on the target value of each of the indicators stored in the exercise log data.

Although the user can understand, by looking at the screen illustrated in FIG. 3, that the user is encouraged to keep in mind to increase the stride-height ratio in the next running, a case is conceivable in which the user forgets, until the next running, the memory of when looking at this screen.

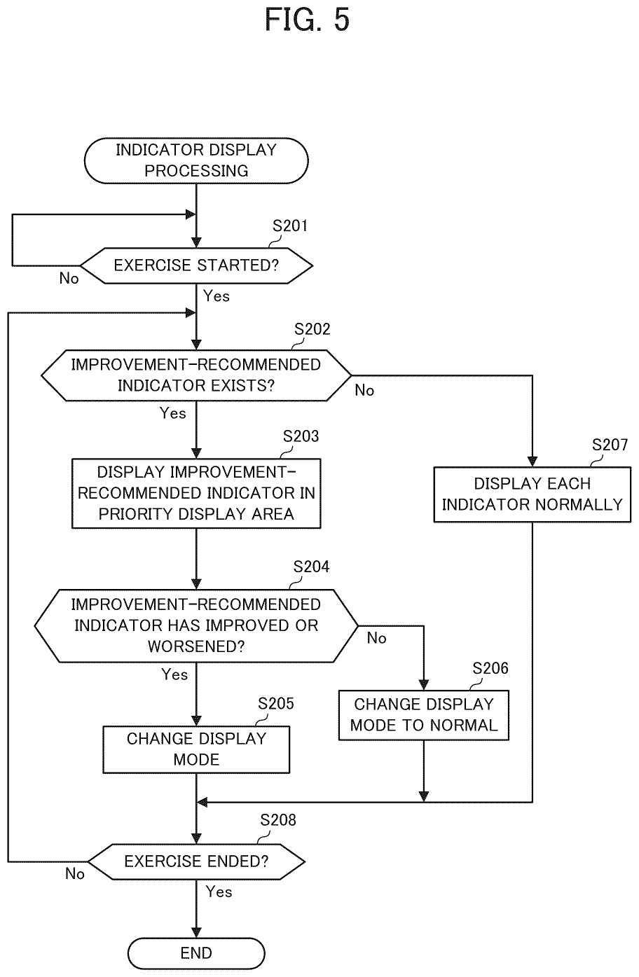

The indicator display processing for enabling checking the improvement-recommended indicator and whether improvement in this indicator is currently achieved even while running is described with reference to FIG. 5. This processing is executable at any timing but is normally executed immediately before the user starts exercising. For example, the user may cause the execution to be started by launching an application that executes the indicator display processing when starting running.

First, the controller 110 determines whether the user has started exercising (for example, running) (step S201). This determination is made by, for example, determining whether the exercise start button 151 is pressed, but this configuration is not limiting. For example, the determination may be made based on whether start of exercise of the user is detected with the measurer 130 or other related element. In the case where exercise is not started (step S201; No), the processing returns to step S201. In the case where exercise has started (step S201; Yes), the controller 110 determines whether the improvement-recommended indicator exists (step S202). The expression “the improvement-recommended indicator exists” indicates that one or more improvement-recommended indicators are extracted in the indicator extraction processing described above.

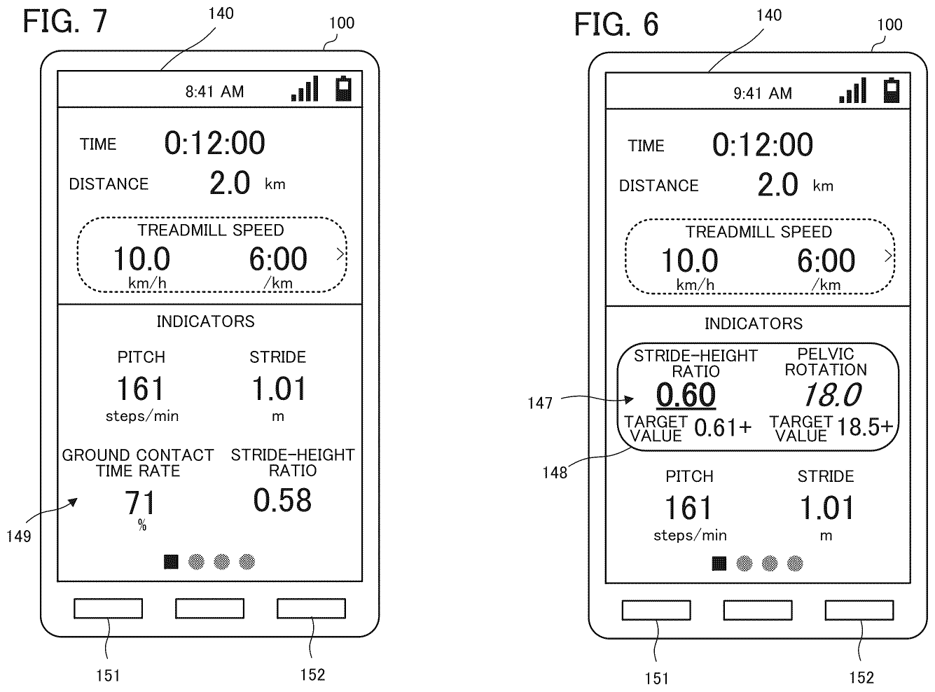

In the case where the improvement-recommended indicator exists (step S202; Yes), the controller 110 causes the improvement-recommended indicator to be displayed in a priority display area 147 of the display 140, as illustrated in FIG. 6 (step S203). The priority display area 147 is an area in the display 140 for displaying information to be shown to the user with priority (an area where the displayed information is easy to see), and displaying the improvement-recommended indicator in the priority display area 147 allows the user to easily check an indicator among the indicators to be improved that has high priority even while the user is exercising. Furthermore, in the case of displaying the improvement-recommended indicator, to make clear that the indicator displayed in the priority display area 147 is the improvement-recommended indicator, the controller 110 may cause the current indicator value (current value) and the target value to be displayed, as illustrated in FIG. 6. Furthermore, as illustrated in FIG. 6, the priority display area 147 may be surrounded by a border 148 so that the surrounded area can be understood as the priority display area 147.

FIG. 6 illustrates an example of displaying two indicators “stride-height ratio” and “pelvic rotation” as the improvement-recommended indicators and displaying the current indicator value and the target value of each of these indicators. This allows the user to easily understand a change in a form for improvement in each of the indicator values. Furthermore, in the case of displaying the target value, displaying not only the target value but also the type (such as “to be increased” (“+”), “to be decreased” (“−”), and “to be kept within a predetermined range” (“=”)) of the corresponding indicator makes the work by the user for score improvement easier. FIG. 6 illustrates an example of displaying “+” to indicate that a value larger than the target value is better than the target value. The number of improvement-recommended indicators, which is two in FIG. 6, depends on the number of improvement-recommended indicators extracted in the indicator extraction processing. Setting one as the number of the improvement-recommended indicators has the advantage that the user can focus only on improvement in this one indicator. Setting two or more as the number of the improvement-recommended indicators has the advantage that the user can select an indicator among the two or more indicators that the user considers easiest with respect to improvement and work for the improvement.

Then the controller 110 determines whether the improvement-recommended indicator has improved or worsened from the indicator value of the most recent exercise (step S204). In this step, the controller 110 compares the indictor score of the corresponding indicator of the most recent exercise with the current indictor score of the corresponding indicator, and determines as “improved” in the case where the current indictor score is higher than the most recent indictor score, and determines as “worsened” in the case where the current indictor score is lower than the most recent indictor score. A configuration may be used in which the criteria for determination has a predetermined range (for example, 10%) and the determination as “improved” is made in the case where the current indictor score is 10% or more higher than the most recent indictor score and the determination as “worsened” is made in the case where the current indicator score is 10% or more lower than the most recent indictor score. This predetermined range may be set differently for the case of “improved” and the case of “worsened”.

In the case where the improvement-recommended indicator is improved or worsened in the indicator value from the most recent exercise (step S204; Yes), the controller 110 changes a display mode for displaying the improvement-recommended indicator (step S205) and proceeds to step S208. The change in the display mode that is made by the controller 110 in step S205 may be any change. One example is to display the indicator value in black normally (by default), whereas displaying the indicator value of the “improved” improvement-recommended indicator in red and displaying the indicator value of the “worsened” improvement-recommended indicator in blue. Instead of (or in addition to) changing the color of the indicator value, the background color of the area in which the indicator is displayed may be changed. Furthermore, as illustrated in FIG. 6, the indicator value of the “improved” improvement-recommended indicator may be displayed with bold font and underline, and the indicator value of the “worsened” improvement-recommended indicator may be displayed in italic font. Note that the determination of “improved” and “worsened” in the present embodiment is based solely on a comparison with the indicator value of the most recent exercise of the user and is made irrespective of whether the target value is achieved. However, in the case where the user has achieved the target value, the controller 110 may similarly perform operations for easy notification of the achievement of the target value to the user, by changing in a display mode for displaying the indicator value achieved the target value, outputting a sound effect (a sound notifying the user that the target value has been achieved) from the sound outputter 160, or the like.

Conversely, in the case where a determination is made in step S204 that the improvement-recommended indicator is neither improved nor worsened (step S204; No), the controller 110 changes the display mode for displaying the improvement-recommended indicator to normal (default setting) (step S206) and proceeds to step S208. In step S206, the controller 110 resets the display mode to display the indicator value in black, for example.

In the case where a determination is made in step S202 that the improvement-recommended indicator does not exist (step S202; No), the controller 110 causes each indicator to be displayed in the display 140 in a normal order (default order) as illustrated in FIG. 7 (step S207) and proceeds to step S208. In this case, since no improvement-recommended indicator exists, various indicators are displayed in the normal order and in a normal display mode. FIG. 7 illustrates displaying “pitch”, “stride”, “ground contact time rate”, and “stride-height ratio” in an indicator display area 149, and this order depends on the default settings.

In step S208, the controller 110 determines whether the user ends exercise (step S208). This determination is made by, for example, determining whether the exercise end button 152 is pressed, but this configuration is not limiting. For example, the determination may be made based on whether end of exercise of the user is detected with the measurer 130 or other related element. In the case where exercise is not yet ended (step S208; No), the processing returns to step S202. In the case where exercise has ended (step S208; Yes), the indicator display processing ends.

With the indicator display processing described above, the electronic device 100 causes, among the indicators to be improved, an indicator with high priority (the improvement-recommended indicator) to be displayed in an easy-to-see area, allowing the user to quickly grasp necessary information out of various types of information. Furthermore, displaying the target value of the improvement-recommended indicator allows the user to obtain a direction on how to improve the exercise of the user. Furthermore, selecting the improvement-recommended indicator in descending order of a degree of contribution to an increase in the score allows the user to work on improvement in the indicator that is easier with respect to the increase in the score. Furthermore, displaying the current indicator value (current value) of the improvement-recommended indicator in real time while the user is exercising enables checking, while exercising (for example, while running), the point for achieving improvement in a level that is conventionally checkable only after the exercise, allowing the user to recognize whether the form of the user is improving. Furthermore, since the display mode of the indicator improved while the user is exercising is changed while the user is exercising, the user can grasp improvement in the indicator in real time, leading to an increase in motivation for improvement in the exercise form.

In the indicator display processing described above, in the case where the indicator value of the improvement-recommended indicator is improved, the controller 110 changes the display mode for displaying the indicator value. However, this configuration is not limiting, and instead of the change in the display mode (or in addition to the change in the display mode), a sound effect or an audio message for notification of improvement in the indicator value may be output from the sound outputter 160. Similarly, in the case where the indicator value of the improvement-recommended indicator is worsened, instead of the change in the display mode (or in addition to the change in the display mode), a sound effect or an audio message for notification that the indicator value is worsened may be output from the sound outputter 160. In the case of sound output, since providing real-time notification of changes in the indicator value is difficult, notification of whether the indicator value of the improvement-recommended indicator is improved or worsened may be provided based on an average value over a predetermined period of time (for example, ten minutes).

In the aforementioned embodiment, the electronic device 100 includes the measurer 130, and thus the measurement values as data related to exercise of the user can be acquired solely by the electronic device 100, that is, with the measurer 130. However, this configuration is not limiting, and a configuration may be used in which the measurer 130 is provided separately from the electronic device 100. For example, a configuration may be used in which processing similar to the processing of the electronic device 100 is executed by an electronic device system that includes a sensor device including the measurer 130 and a communicator and an electronic device having the configuration of the electronic device 100 with the measurer 130 removed and a communicator added (sensor-unequipped electronic device).

In such an electronic device system, the controller 110 of the sensor-unequipped electronic device acquires via the communicator the measurement values measured with the measurer 130 of the sensor device and thus can execute the indicator extraction processing, the indicator display processing, and other related processing. In the case of using this electronic device system, the user can, for example, wear the sensor device on the waist and sensor-unequipped electronic device sensor on the arm to achieve both acquisition of high-accuracy measurement values and display of the indicator and other information in an easy-to-see manner.

Furthermore, a configuration may be used in which earphones as the sound outputter 160 are provided as a separate ear device that communicates with the electronic device via Bluetooth (registered trademark) or other short-range wireless technology. Furthermore, a configuration may be used in which the display 140 is provided as a separate display device that communicates with the electronic device via a short-range wireless technology. As described above, the electronic device 100 need not include all the elements thereof in one single housing, and any functional element (such as the measurer 130, the display 140, the operation inputter 150, the sound outputter 160, and the like) may be provided as a separate body as appropriate.

Furthermore, although the aforementioned embodiment mainly describes an example in which the exercise of the user is running, the exercise of the user handled by the electronic device 100 is not limited to running and may be walking or cycling. The indicators and the evaluation items to be derived and displayed may be freely changed depending on the type of exercise, and the like.

Furthermore, the electronic device 100 is not limited to a wearable device, and can be achieved by any computer that can acquire the measurement values with the measurer 130, such as a smartphone, a portable tablet, personal computer (PC), or the like. Specifically, in the aforementioned embodiment, the description is made assuming that the program for the indicator extraction processing, the indicator display processing, and other related processing to be executed by the controller 110 is pre-stored in the storage 120. However, storing and distributing the program on a non-transitory computer-readable recording medium such as a compact disc read-only memory (CD-ROM), a digital versatile disc (DVD), a magneto-optical, disc (MO), a memory card, and a USB memory, and reading out and installing this program on a computer enables achieving a computer that is capable of executing the various processings described above.

Furthermore, the program can be superimposed on a carrier wave and applied via a communication medium such as the internet. For example, the program may be posted to and distributed via a bulletin board system (BBS) on a communication network. Moreover, a configuration may be used in which the various processings described above are executed by starting the program and, under the control of the operating system (OS), executing the program in the same manner as other applications/programs.

Additionally, the controller 110 may be achieved solely by a freely-selected processor unit, such as a single processor, a multiprocessor, a multi-core processor, or the like, or may be achieved by a combination of such freely-selected processor unit and processing circuitry such as an application specific integrated circuit (ASIC), a field-programmable gate array (FPGA), or the like.

The foregoing describes some example embodiments for explanatory purposes. Although the foregoing discussion has presented specific embodiments, persons skilled in the art will recognize that changes may be made in form and detail without departing from the broader spirit and scope of the invention. Accordingly, the specification and drawings are to be regarded in an illustrative rather than a restrictive sense. This detailed description, therefore, is not to be taken in a limiting sense, and the scope of the invention is defined only by the included claims, along with the full range of equivalents to which such claims are entitled.

1. An electronic device to be equipped by a user for acquiring data related to exercise of the user, the electronic device comprising:
an acquirer to acquire a measurement result that is a result of measurement of each of indicator values of a plurality of indicators related to the exercise;
a storage to store the measurement result acquired by the acquirer;
a display to display information based on the measurement; and
a controller to, in response to start of new measurement, based on one or more indicator values that are included in the indicator values of the plurality of indicators included in the measurement result of most recent measurement stored in the storage and that satisfy a predetermined condition, determine a combination of two or more indicators of the plurality of indicators that are to be displayed on the display.

2. The electronic device according to claim 1, wherein
the controller
sets a target value of each of the indicators determined to be displayed on the display, and
causes the set target value to be displayed on the display.

3. The electronic device according to claim 2, wherein
the controller
calculates a predefined score based on the indicator values of the plurality of indicators included in the measurement result of the most recent measurement, and
selects a predetermined number of the indicators from the plurality of indicators in descending order of a degree of contribution to an increase in the score, and causes the selected one or more indicators to be displayed on the display.

4. The electronic device according to claim 2, wherein
the controller
selects a predetermined number of the indicators from the plurality of indicators in descending order of a difference between the target value and the corresponding indicator value of the plurality of indicator values included in the measurement result of the most recent measurement, and causes the selected one or more indicators to be displayed on the display.

5. The electronic device according to claim 3, wherein
the controller
causes a current value to be displayed on the display, the current value being a value that is currently acquired by the acquirer for each of the selected one or more indicators.

6. The electronic device according to claim 5, wherein
the controller
causes an indicator with the current value that is improved with respect to the most recent measurement result to be displayed on the display with a change in a display mode for displaying the current value.

7. The electronic device according to claim 5, wherein
the display includes a priority display area for displaying information to be shown to the user with priority, and
the controller
causes the selected one or more indicators to be displayed in the priority display area of the display.

8. The electronic device according to claim 1, wherein
the controller selects a predetermined number of the one or more indicator values that satisfy the predetermined condition, and causes the selected one or more indicators to be displayed on the display, and
the predetermined number is a predetermined constant or a number that is freely determined by the user.

9. An information display method executable with a controller included in an electronic device that includes an acquirer to be equipped by a user for acquiring data related to exercise of the user and a display, the information display method comprising:
by the controller,
storing, in a storage, a measurement result that is a result of measurement of each of indicator values of a plurality of indicators related to the exercise; and
in response to start of new measurement, based on one or more indicator values that are included in the indicator values of the plurality of indicators included in the measurement result of most recent measurement stored in the storage and that satisfy a predetermined condition, changing a combination of two or more indicators of the plurality of indicators that are to be displayed on the display.

10. The information display method according to claim 9, further comprising:
by the controller,
setting a target value of each of the indicators determined to be displayed on the display, and
causing the set target value to be displayed on the display.

11. The information display method according to claim 10, further comprising:
by the controller,
calculating a predefined score based on the indicator values of the plurality of indicators included in the measurement result of the most recent measurement; and
selecting a predetermined number of the indicators from the plurality of indicators in descending order of a degree of contribution to an increase in the score, and causing the selected one or more indicators to be displayed on the display.

12. The information display method according to claim 10, further comprising:
by the controller,
selecting a predetermined number of the indicators from the plurality of indicators in descending order of a difference between the target value and the corresponding indicator value of the plurality of indicator value included in the measurement result of the most recent measurement, and causing the selected one or more indicators to be displayed on the display.

13. A non-transitory recording medium storing a program readable by a computer of an electronic device that includes an acquirer to be equipped by a user for acquiring data related to exercise of the user and a display, the program causing the computer to:
store, in a storage, a measurement result that is a result of measurement of each of indicator values of a plurality of indicators related to the exercise, and
in response to start of new measurement, based on one or more indicator values that are included in the indicator values of the plurality of indicators included in the measurement result of most recent measurement stored in the storage and that satisfy a predetermined condition, determine a combination of two or more indicators of the plurality of indicators that are to be displayed on the display.
